(19) United States
(12) Patent Application Publication
(10) Pub. No.: US 2016/0121108 A1
(43) Pub. Date: May 5, 2016

(54) BEAUTY INSTRUMENT

(71) Applicant: ARTISTIC&CO.CO.,LTD., Ichinomiya-city, Aichi (JP)

(72) Inventors: Hideki KONDO, Ichinomiya-city, Aichi (JP); Yeon Goog s, Anyang-si, Gyeonggi-do (KR)

(73) Assignee: ARTISTIC&CO.CO.,LTD., Ichinomiya-city, Aichi, OT (JP)

(21) Appl. No.: 14/896,332

(22) PCT Filed: Oct. 29, 2014
(86) PCT No.: PCT/JP2014/078716

§ 371 Date: Dec. 4, 2015

(30) Foreign Application Priority Data
Nov. 11, 2013 (JP) 2013-232717

(51) Int. Cl. A61N 1/32; A61N 7/00; A61N 1/06; A61N 1/04; A61N 5/06

(52) U.S. Cl. CPC A61N 1/328; A61N 1/0476; A61N 5/0616; A61N 1/06; A61N 7/00; A61N 2005/0651; A61N 2005/0663; A61N 2007/0034

(57) ABSTRACT

A beauty instrument includes a plurality of electrodes separated into a first electrode group and a second electrode group that are continuous with each other. The electrode groups are controlled so that a current supply state and a current supply suspension state are alternated between the two groups every predetermined period of time. An operation of four electrodes of each of the first electrode group and the second electrode group can be so controlled that a positive electric charge of one pair of the electrodes and a negative electric charge of the other pair of the electrodes are alternately replaced with each other with a diagonal line connecting the diagonally disposed two positive electrodes to each other crossing with a diagonal line connecting the diagonally disposed two negative electrodes to each other.

### TECHNICAL FIELD

The present invention relates to a beauty instrument for bringing about a cosmetic effect such as a massage effect for a user by applying a weak electric current to user's skin with the beauty instrument in close contact with the user's skin.

### BACKGROUND ART

Beauty instruments for providing the user's skin with the cosmetic effect such as a massage effect by applying a weak electric current to the user's skin with the beauty instrument in close contact therewith are known. It is known that various methods are used for beauty instruments to bring about the cosmetic effect for the skin. A method of heating or cooling the skin, sucking the skin, a method of applying steam or mist to the skin, a method of imparting vibration to the skin by means of ultrasonic waves, a method of directly imparting vibration to the skin by means of a vibrator, a method of irradiating the skin with light by means of an LED or the like, and a method of applying a weak electric current to the skin are known. Of these conventional arts, a beauty instrument to be used for an iontophoresis method attracts attention in recent years. The beauty instrument applies a weak electric current to user's skin (see for example, patent document 1).

### PRIOR ART DOCUMENT

### Patent Document

Patent document 1: Japanese Patent Application Laid-Open Publication Number 2005-253501 (FIG. 1 and so on)

### SUMMARY OF THE INVENTION

### Problems to be Solved by the Invention

But in using the above-described conventional beauty instrument which applies a weak electric current to the user's skin, the user feels that there is a poor close contact between the electrode of the beauty instrument and the user's skin. In addition, because only a certain bodily sensation continues during the use of the beauty instrument, the beauty instrument has a low level of massage effect. Further the user feels uncomfortable in the use of the beauty instrument for the user's skin. Furthermore there is a fear that the user feels monotonous in the use of the beauty instrument. As described above, the conventional beauty instruments have a limitation in allowing the user to feel that the beauty instrument is in close contact with the user's skin to a higher extent than conventional beauty instruments.

The present invention has been developed to solve the problems of the above-described conventional beauty instruments. Therefore it is object of the present invention to provide a beauty instrument which allows a user to feel that the beauty instrument is in close contact with the user's skin to a higher extent than conventional beauty instruments and brings about a rhythmical massage effect.

### Means for Solving the Problems

Means for achieving the above-described object is as described below.

(1) A beauty instrument comprising a plurality of electrodes and being capable of applying high-frequency waves to user's skin from the electrodes thereof, wherein a plurality of the electrodes is continuously arranged, and an operation of a plurality of the electrodes can be so controlled that a current supply state and a current supply suspension state are alternated among a plurality of the electrodes every predetermined period of time.

(2) A beauty instrument comprising a plurality of electrodes capable of applying high-frequency waves to user's skin from the electrodes thereof, wherein a plurality of the electrodes is arranged by separating the electrodes into a plurality of electrode groups with the electrodes being continuous with each other, and an operation of the electrode groups can be so controlled that a current supply state and a current supply suspension state are alternated among a plurality of the electrode groups every predetermined period of time.

### BRIEF DESCRIPTION OF THE DRAWINGS

FIG. 1 is a front view of a beauty instrument of an embodiment of the present invention.

FIG. 2 is a right side view of the beauty instrument shown in FIG. 1.

FIG. 3 is a rear view of the beauty instrument shown in FIG. 1.

FIG. 4 is a front view of a head part of the beauty instrument shown in FIG. 1.

FIG. 5 is block diagram of an electric circuit of the beauty instrument shown in FIG. 1.

FIG. 6 is an image view of electrodes of the head part.

FIG. 7 is an image view of the electrodes of the head part.

FIG. 8 is an image view of the electrodes of the head part.

FIG. 9 is a perspective view of a beauty instrument of another embodiment of the present invention.

FIG. 10 is a main part-depicted front view of a beauty instrument of still another embodiment of the present invention.

FIG. 11 is a main part-depicted front view of a beauty instrument of still another embodiment of the present invention.

FIG. 12 is a main part-depicted front view of a beauty instrument of still another embodiment of the present invention.

FIG. 13 is a main part-depicted front view of a beauty instrument of still another embodiment of the present invention.

### MODE FOR CARRYING OUT THE INVENTION

The embodiments of the present invention are described below. The embodiments of the present invention are as described below.

(1) A beauty instrument comprising a plurality of electrodes and being capable of applying high-frequency waves to user's skin from the electrodes thereof, wherein a plurality of the electrodes is continuously arranged, and an operation of a plurality of the electrodes can be so controlled that a current supply state and a current supply suspension state are alternated among a plurality of the electrodes every predetermined period of time.

By using the high-frequency waves, the beauty instrument allows a user to feel that the beauty instrument is in close contact with the user's skin to a higher extent than conventional beauty instruments. The beauty instrument is provided with a plurality of the electrodes. The current supply state and the current supply suspension state are alternated every predetermined period of time among a plurality of the electrodes (for example, initially, the first electrode is placed in the current supply state, whereas the second electrode is placed in the current supply suspension state. Thereafter the current supply state and the current supply suspension state are alternated between the first electrode and the second electrode every 0.5 seconds. Alternatively, initially, the first electrode and the second electrode are placed in the current supply state, whereas a third electrode is placed in the current supply suspension state. Thereafter the current supply state and the current supply suspension state are alternated among the first electrode, the second electrode, and the third electrode every 0.5 seconds. Alternatively, the first electrode is placed in the current supply state, whereas the current supply state and the current supply suspension state are alternated with each other in the second electrode every 0.5 seconds.) Thus in addition to the above-described user's feeling that the beauty instrument is in close contact with the user's skin to a higher extent than conventional beauty instruments, the user feels a rhythmical massage effect that the user's skin is tapped alternately by different electrodes because the current supply state and the current supply suspension state are alternated among different electrodes.

(2) A beauty instrument comprising a plurality of electrodes capable of applying high-frequency waves to user's skin from the electrodes thereof, wherein a plurality of the electrodes is arranged by separating the electrodes into a plurality of electrode groups with the electrodes being continuous with each other, and an operation of the electrode groups can be so controlled that a current supply state and a current supply suspension state are alternated among a plurality of the electrode groups every predetermined period of time.

By using the high-frequency waves, the beauty instrument allows the user to feel that the beauty instrument is in close contact with the user's skin to a higher extent than conventional beauty instruments. The beauty instrument is provided with a plurality of the electrode groups. The current supply state and current supply suspension state are alternated every predetermined period of time among a plurality of the electrode groups each consisting of a plurality of electrodes (for example, initially, the first electrode group is placed in the current supply state, whereas the second electrode group is placed in the current supply suspension state. Thereafter the current supply state and the current supply suspension state are alternated between the first electrode group and the second electrode group every 0.5 seconds. Alternatively, initially, the first electrode group and the second electrode group are placed in the current supply state, whereas a third electrode group is placed in the current supply suspension state. Thereafter the current supply state and the current supply suspension state are alternated among the first electrode group, the second electrode group, and the third electrode group every 0.5 seconds. Alternatively, the first electrode group is placed in the current supply state, whereas the current supply state and the current supply suspension state are alternated with each other in the second electrode group every 0.5 seconds.) Thereby the user can feel a rhythmical massage effect that the user's skin is tapped alternately by different electrodes in addition to the above-described user's feeling that the beauty instrument is in close contact with the user's skin to a higher extent than conventional beauty instruments. Because the user can feel a rhythmical massage effect that the user's skin is tapped alternately by different electrodes, the user can obtain the massage effect in each wide range in which a plurality of the electrodes is arranged. Thereby the user can clearly recognize the massage effect given by different portions of the beauty instrument.

(3) The beauty instrument according to claim 1, wherein a number of a plurality of the electrodes is set to 4n (n=1 or more), and four electrodes of each electrode group are disposed at positions of apexes of a quadrangle, and an operation of the four electrodes disposed at the positions of the apexes of the quadrangle can be so controlled that a positive electric charge of one pair of the electrodes and a negative electric charge of the other pair of the electrodes are alternately replaced with each other with a diagonal line connecting the positive diagonally disposed two electrodes to each other crossing with a diagonal line connecting the negative diagonally disposed two electrodes to each other.

By arranging a plurality of the electrodes in the above-described way and controlling the operation of the electrodes in such a way that the positive electric charge of one pair of the electrodes and the negative electric charge of the other pair of the electrodes are alternately replaced with each other, the beauty instrument allows the user to feel that the beauty instrument is in close contact with the user's skin to a higher extent in the entire range where the four electrodes are arranged. Further by alternating the current supply state and the current supply suspension state with each other, the beauty instrument allows the user to expect a synergistic effect of enhancing the massage effect.

(4) The beauty instrument according to claim 2, wherein a number of a plurality of electrodes of each of the electrode groups is set to four, and the four electrodes are disposed at positions of apexes of a quadrangle, and an operation of the four electrodes disposed at the positions of the apexes of the quadrangle can be so controlled that a positive electric charge of one pair of the electrodes and a negative electric charge of the other pair of the electrodes are alternately replaced with each other with a diagonal line connecting the diagonally disposed two positive electrodes to each other crossing with a diagonal line connecting the diagonally disposed two negative electrodes to each other.

By arranging the electrodes of each electrode group in the above-described way and controlling the operation of the electrodes in such a way that the positive electric charge of one pair of the electrodes and the negative electric charge of the other pair of the electrodes are alternately replaced with each other, the beauty instrument allows the user to feel that the beauty instrument is in close contact with the user's skin to a higher extent in the entire range where the four electrodes are disposed. Further by alternating the current supply state and the current supply suspension state among the continuously arranged electrode groups, the beauty instrument allows the user to expect a synergistic effect of enhancing the massage effect.

(5) The beauty instrument according to claim 2 or 4 formed substantially cylindrically, and at least two of the electrode groups are adjacently and continuously arranged along a longitudinal direction of the beauty instrument.

By adjacently and continuously arranging at least two of the electrode groups along the longitudinal direction of the beauty instrument formed substantially cylindrically, the cylindrical beauty instrument formed compactly in its entire size makes it possible to decrease the space necessary for disposing the electrode groups at the electrode arranging portion and allows the user to feel a rhythmic massage effect that the user's skin is alternately tapped by different electrode groups.

(6) The beauty instrument according to claim 1, wherein a plurality of the electrodes is linearly arranged, and an operation of the linearly arranged electrodes can be so controlled that the electrodes are sequentially placed in a current supply state in a direction from the electrode disposed at one-end side of the linearly arranged electrodes toward the electrode disposed at the other-end side thereof and thereafter sequentially placed in a current supply suspension state.

By linearly arranging a plurality of the electrodes as a straight line, a curve or the like, sequentially placing the linearly arranged electrodes in the current supply state in the direction from the electrode positioned at the one-end side of the linearly arranged electrodes toward the electrode positioned at the other-end side thereof, and thereafter sequentially placing the electrodes in the current supply suspension state, the user can obtain a massage effect which flows from a portion of the user's skin corresponding to the one-end side of the linearly arranged electrodes to a portion of the user's skin corresponding to the other-end side thereof. The mode of “sequentially placing the electrodes in the current supply state and thereafter sequentially placing the electrodes in the current supply suspension state” includes an operation of sequentially supplying an electric current to all the electrodes and thereafter sequentially placing all the electrodes in the current supply suspension state or an operation of supplying the electric current to one electrode, thereafter placing the electrode in the current supply suspension state, and thereafter supplying the electric current to remaining electrodes subsequent thereto. The above-described mode includes the operation of sequentially placing the electrodes in the current supply state and thereafter in the current supply suspension state.

(7) The beauty instrument according to claim 1, wherein a plurality of the electrodes is arranged in a direction from a center of an electrode arranging portion toward an outer periphery thereof, and an operation of a plurality of the arranged electrodes can be so controlled that the arranged electrodes are sequentially placed in a current supply state in a direction from the electrodes disposed at a center of the arranged electrodes toward the electrodes disposed along an outer periphery thereof and thereafter sequentially placed in a current supply suspension state.

By arranging a plurality of the electrodes in the direction from the center of the electrode arranging portion toward the outer periphery thereof, sequentially placing a plurality of the arranged electrodes in the current supply state in the direction from the electrodes disposed at the center of the arranged electrodes toward the electrodes disposed along the outer periphery thereof, and thereafter sequentially placing the electrodes in the current supply suspension state, the user can obtain a massage effect which spreads from a portion of the user's skin corresponding to the center of the arranged electrodes toward a portion of the user's skin corresponding to the outer peripheral side thereof. The mode of “sequentially placing the electrodes in the current supply state and thereafter sequentially placing the electrodes in the current supply suspension state” includes an operation of sequentially supplying the electric current to all the arranged electrodes in the direction from the electrodes disposed at the center of all the arranged electrodes toward the electrodes disposed along the outer periphery thereof and thereafter sequentially placing all the arranged electrodes in the current supply suspension state in the direction from the electrodes disposed at the center of all the arranged electrodes toward the electrodes disposed along the outer periphery thereof or an operation of supplying the electric current to the electrodes disposed at the center of all the arranged electrodes, thereafter placing the electrodes in the current supply suspension state, and thereafter supplying the electric current to the electrodes disposed outward from and adjacent to the electrodes disposed at the center of all the arranged electrodes. That is, the above-described mode includes the operation of sequentially placing the remaining electrodes in the current supply state and the current supply suspension state.

(8) The beauty instrument according to claim 1, wherein a plurality of the electrodes is arranged in a direction from an outer periphery of an electrode arranging portion toward a center thereof, and an operation of a plurality of the arranged electrodes can be so controlled that the arranged electrodes are sequentially placed in a current supply state in a direction from the electrodes disposed along an outer periphery of the arranged electrodes toward the electrodes disposed at a center thereof and thereafter sequentially placed in a current supply suspension state.

By arranging a plurality of the electrodes in the direction from the outer periphery of the electrode arranging portion toward the center thereof, sequentially placing a plurality of the arranged electrodes in the current supply state in the direction from the electrodes disposed along the outer periphery thereof toward the electrodes disposed at the center thereof, and thereafter sequentially placing the electrodes in the current supply suspension state, the user can obtain a massage effect which flows from a portion of the user's skin corresponding to the outer peripheral side of the arranged electrodes toward a portion of the user's skin corresponding to the center thereof. The mode of “sequentially placing the electrodes in the current supply state and thereafter sequentially placing the electrodes in the current supply suspension state” includes an operation of sequentially supplying the electric current to all the electrodes in the direction from the electrodes disposed along the outer periphery of the arranged electrodes toward the electrodes disposed at the center thereof and thereafter sequentially placing all the electrodes in the current supply suspension state in the direction from the electrodes disposed along the outer periphery of the arranged electrodes toward the electrodes disposed at the center thereof or an operation of supplying the electric current to the electrodes disposed along the outer periphery of the arranged electrodes and thereafter placing the electrodes in the current supply suspension state, and thereafter supplying the electric current to the electrodes disposed inward from and adjacent to the electrodes disposed along the outer periphery thereof. That is, the above-described mode includes the operation of sequentially placing the remaining electrodes in the current supply state and the current supply suspension state.

(9) The beauty instrument according to any one of claims 1 through 8, wherein an operation of the electrodes can be so controlled that a current supply state and a current supply suspension state are alternated among the electrodes at time intervals of 0.20 seconds to 3.00 seconds.

By alternating the current supply state and the current supply suspension state among the electrodes at the time intervals of 0.20 seconds to 3.00 seconds, the beauty instrument allows the user to have a clear feeling that the user alternately receives a massage effect from different electrodes.

(10) The beauty instrument according to any one of claims 1 through 8, wherein an operation of the electrodes can be so controlled that a current supply state and a current supply suspension state are alternated among the electrodes at time intervals of 0.25 seconds to 1.00 second.

By alternating the current supply state and the current supply suspension state among the electrodes at the time intervals of 0.25 seconds to 1.00 second, it is possible to alternate the current supply state and the current supply suspension state among the electrodes at an early time, namely, from a rise time in an initial supply of the electric current to the electrodes. Therefore the beauty instrument allows the user to have a clear feeling that the user alternately receives a massage effect from different electrodes from the rise time in the initial supply of the electric current to the electrodes.

(11) The beauty instrument according to any one of claims 1 through 10 comprising an LED capable of irradiating user's skin with light.

In addition to the above-described effect, the beauty instrument provides various improved effects for the skin owing to the light emitted by the LED.

(12) The beauty instrument according to claim 11, wherein the LED can be so controlled that simultaneously with a supply of an electric current from the electrodes to the LED, the LED is capable of irradiating user's skin with light.

By so controlling the LED that the LED is capable of irradiating the user's skin with light simultaneously with the supply of the electric current to the LED, it is possible to achieve an effect synergistic with the effect for the skin to be brought about owing to the action of high frequency waves and shorten a treatment period of time.

(13) The beauty instrument according to claim 11 or 12, wherein the LED consists of a blue light-emitting diode, a red light-emitting diode, and a yellow light-emitting diode.

It is possible to bring about the effect of softening the roughness of the user's skin and giving exhilaration to the skin owing to the action of the wavelength of the blue light-emitting diode, the effect of giving firmness to the user's skin and allowing the skin to be shiny and elastic owing to the action of the wavelength of the red light-emitting diode, and the effect of allowing the skin to be bright and transparent owing to the action of the wavelength of the yellow light-emitting diode. In addition to the above-described effects to be brought about by the action of the electrode, the beauty instrument is capable of synergistically bringing about a desired beauty effect by appropriately selecting these LEDs according to user's purpose.

(14) The beauty instrument according to any one of claims 1 through 13 further comprising an electrode to which an electric current having a frequency of about 400 kHz to about 800 kHz at which a high deep part heating effect is obtained.

(15) The beauty instrument according to any one of claims 1 through 14 further comprising an ultrasonic generator capable of generating ultrasonic waves having an output frequency of about 43 kHz to about 54 kHz and a vibration frequency of about 70 Hz to about 90 Hz for generating cavitation.

The beauty instruments of the embodiments of the present invention allow the user to feel that the beauty instrument is in close contact with the user's skin to a higher extent than conventional beauty instruments owing to the use of high frequency waves. The beauty instrument is provided with a plurality of electrodes or a plurality of electrode groups each consisting of a plurality of electrodes. The operation of the electrodes or the electrode groups is so controlled that the current supply state and the current supply suspension state are alternated among the electrodes or among the electrode groups every predetermined period of time. Thereby in addition to the above-described feeling of the close contact between the beauty instrument and the user's skin, the user can also feel a rhythmical massage effect that the user's skin is alternately tapped by different electrodes or different electrode groups.

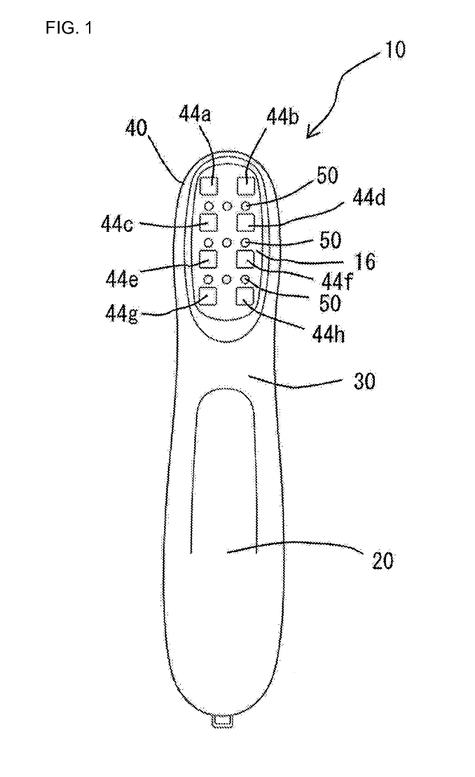

The beauty instruments of the present invention are described below with reference to the embodiments shown in the drawings. As shown in FIGS. 1 through 3, a beauty instrument 10 of an embodiment of the present invention is vertically long. The thickness of the beauty instrument in a front-to-back direction is a little smaller than its width in a left-to-right direction. The beauty instrument 10 is elliptic in a front view and entirely cylindrical. The beauty instrument 10 is intended to allow a massage effect to be brought about for user's skin to a high extent by supplying a weak electric current to the user's skin from electrodes 44 provided at a head part 40.

The beauty instrument 10 is provided with the head part 40 having the electrodes 44 to be described later at an upper part thereof and a gripping part 20 the user grips in using the beauty instrument 10 at a lower part thereof. An intermediate part 30 disposed between the head part 40 and the gripping part 20 is formed small in the width thereof in a front view of the beauty instrument 10. In an entire configuration of the beauty instrument 10, the intermediate part 30 is constricted like a long and narrow gourd.

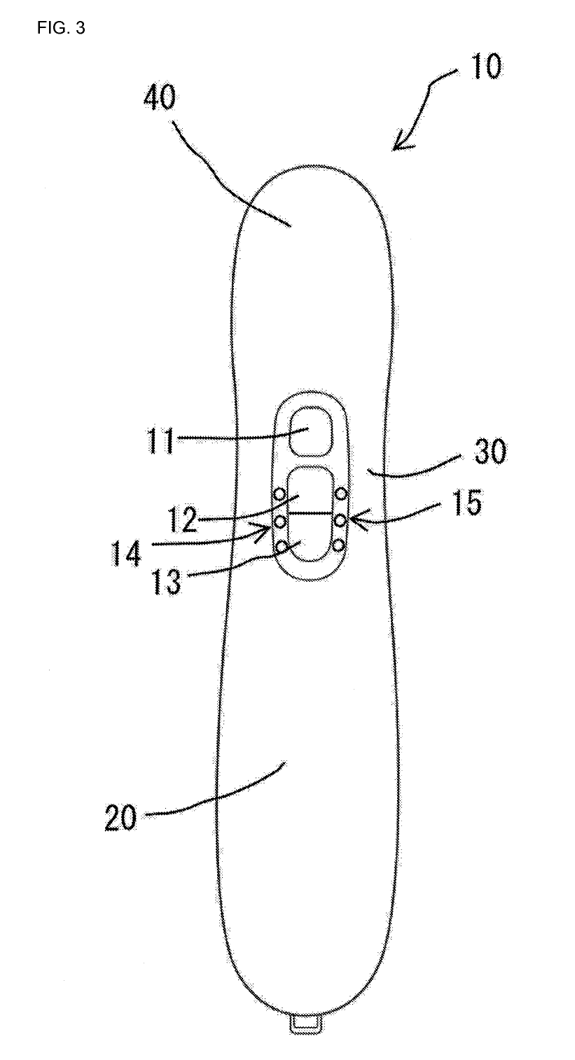

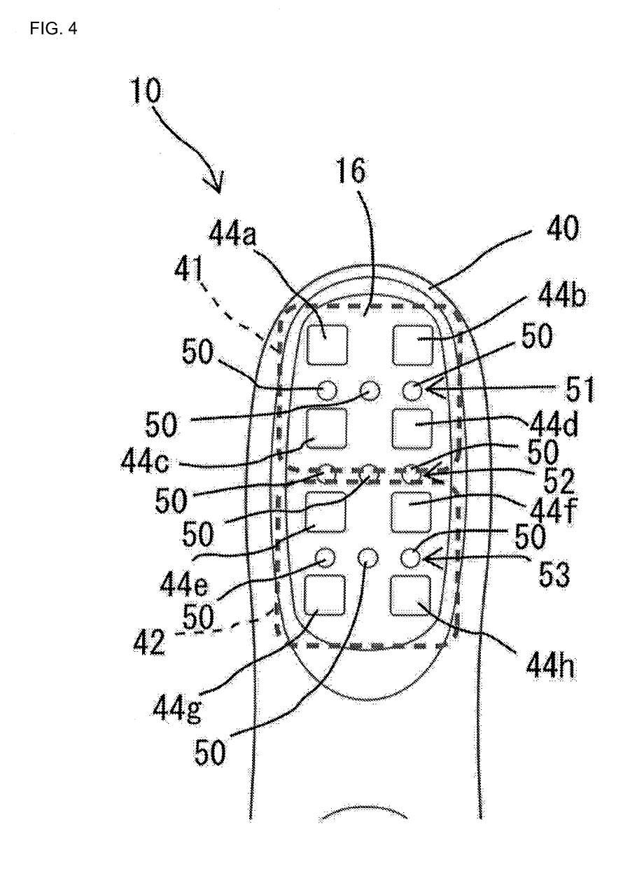

As shown in FIG. 3, the intermediate part 30 of the beauty instrument 10 is provided with three operation buttons disposed at a rear side thereof. The operation buttons are an LED button 11, a power button 12, and a mode button 13 arranged in this order from above. The LED button 11 is the operation button for operating an action of lighting an LED described later. The power button 12 is the operation button for operating ON-OFF of a power supply for the beauty instrument 10 and the level of an output of each electrode 44. The mode button 13 is the operation button, provided at the head part 40, for performing an operation of altering modes of electric currents flowing from the electrodes 44, as described later. Although not shown in the drawings, the beauty instrument 10 can be connected to an AC power supply by means of an AC adaptor.

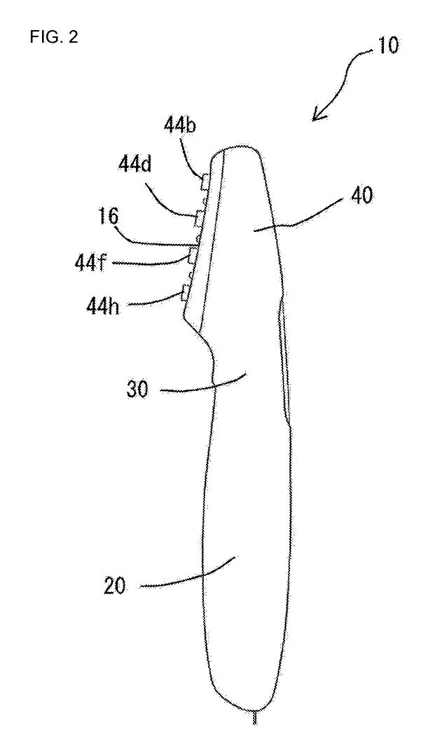

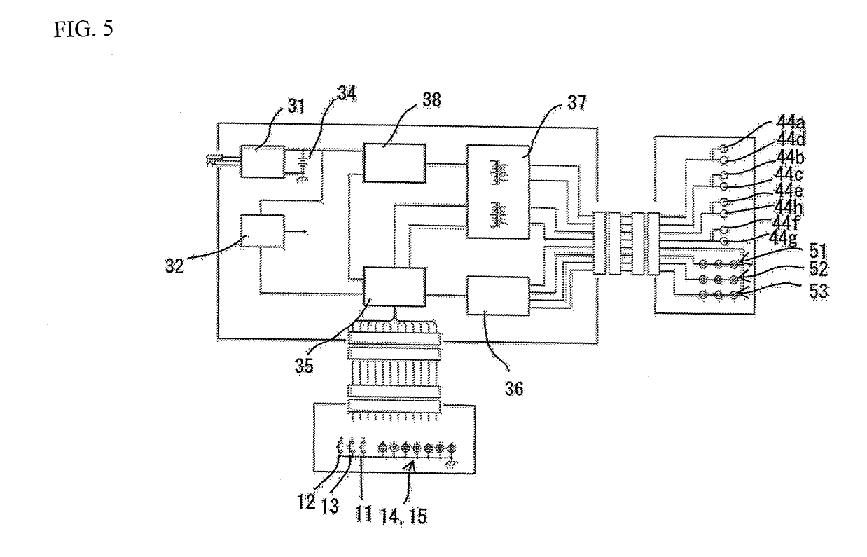

As shown in FIGS. 1 and 2, an elliptic electrode arranging portion 16 elliptic in a front view is formed planarly at a front side of the head part 40. As described later, the electrode arranging portion 16 has eight electrodes 44 and nine LEDs 50 arranged thereon. As shown in FIG. 5, the beauty instrument 10 incorporates an electronic circuit composed of a power supply substrate 31 for controlling the operation of each electrode and that of each LED 50, a converter 32, a microcomputer 35, an LED driver 36, a power booster 37, and an RF power controller 38. The beauty instrument 10 also incorporates a rechargeable battery 34. Thus it is possible to use the beauty instrument 10 charged by the AC power supply by removing the AC adaptor therefrom.

The details of the head part 40 are described below. As described above, eight electrodes 44 and nine LEDs 50 are disposed at the electrode arranging portion 16 of the head part 40. The electrodes 44 are arranged in four columns by two rows by spacing the electrodes 44 at regular intervals in a vertical direction. A first electrode group 41 is composed of electrodes 44a, 44b, 44c, and 44d of these electrodes 44 disposed at the positions of the apexes of a square in a region near a distal end of the head part 40. A second electrode group 42 is composed of electrodes 44e, 44f, 44g, and 44h disposed at the positions of the apexes of a square in a region near a proximal end of the head part 40. The beauty instrument 10 of the present invention is intended to enhance a beauty effect for the user's skin by supplying a weak electric current of 90 kHz thereto from each of the electrodes 44.

As described above, the first electrode group 41 is composed of the electrodes 44a, 44b, 44c, and 44d disposed at the positions of the apexes of the square. The electrodes 44a, 44b, 44c, and 44d are formed in an identical configuration. Each of the electrodes is formed as a square in a front view. The length of the side of the square is set to about 8 mm. Corners of each of the electrodes as viewed from the front side thereof are chamfered at R2.5. As shown in FIG. 2, each of the electrodes 44 is projected slightly forward from the surface of the electrode arranging portion 16 to form each electrode as a thin plate. The height of each electrode from the surface of the electrode arranging portion 16 is set to about 2 mm. Corners of each electrode as viewed from a side surface thereof are chamfered at R1. In the electrodes 44a, 44b, 44c, and 44d, the interval between one electrode disposed at the position of one apex of the square and the other electrodes disposed at the positions of the adjacent apexes thereof is set to about 6 mm. As with the first electrode group 41, the configurations of the second electrode group 42 are identical to each other, and the intervals between the adjacent electrodes are equal to each other. The interval between the electrode 44c of the first electrode group 41 and the electrode 44e of the second electrode group 42 and the interval between the electrode 44d of the first electrode group 41 and the electrode 44f of the second electrode group 42 are also set to about 6 mm.

Each of three LED arrays 51 through 53 consisting of three LEDs 50 respectively is arranged between the vertically adjacent electrodes arranged in four columns. Thus nine LEDs in total are arranged at the electrode arranging portion. The beauty instrument 10 is intended to enhance the beauty effect to be described later owing to the action of the LEDs 50. The uppermost first LED array 51 is disposed laterally between the electrodes 44a, 44b and the electrodes 44c, 44d. Each of the three LEDs 50 of the LED array 51 consists of a blue light-emitting diode. The second LED array 52 is disposed laterally between the electrodes 44c, 44d and the electrodes 44e, 44f. Each of the three LEDs 50 of the LED array 52 consists of a red light-emitting diode. The lowermost third LED array 53 is disposed laterally between the electrodes 44e, 44f and the electrodes 44g, 44h. Each of the three LEDs 50 of the LED array 53 consists of a yellow light-emitting diode. As described above, the LED arrays 51 through 53 are arranged in the lateral direction of the beauty instrument 10 by disposing the LED arrays 51 through 53 within the lateral range of the electrodes 44 arranged vertically in two rows. As described above, nine LEDs 50 in total are arranged in three columns by three rows at the electrode arranging portion 16 of the head part 40.

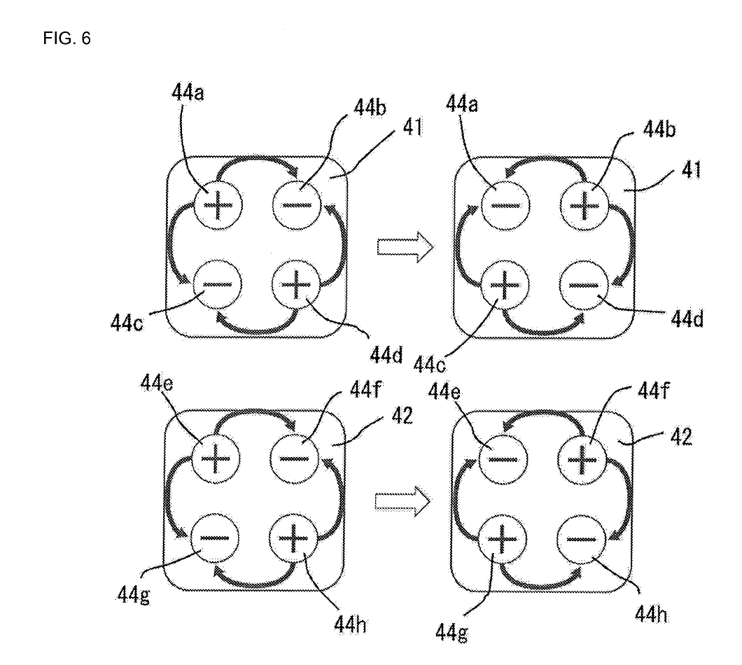

The operation and effect of the electrode 10 constructed as described above are described below. As described above, it is possible to flow the electric current of 90 kHz from each electrode 44 to the user's skin. Normally, when the electric current is applied to the user's skin, the lower the frequency of the electric current becomes, the higher the impedance becomes at a portion of contact between the electrode and the skin, and a temporal rate in which the user feels an electrical signal tends to become long. As a result, stimulus to be given to the skin is liable to become high. By using a high frequency of 90 kHz, as described above, the beauty instrument 10 is intended to decrease the stimulus to be given to the skin. As shown in FIG. 6, the operation of the four electrodes of each of the electrode groups 41 and 42 can be so controlled that a positive electric charge of one pair of the electrodes and a negative electric charge of the other pair of the electrodes (for example, the first electrode group 41 consists of a combination of the positive electrodes 44a and 44d and the negative electrodes 44b and 44c) are alternately replaced with each other 90,000 times per second with a diagonal line connecting the diagonally disposed two positive electrodes to each other crossing with a diagonal line connecting the diagonally disposed two negative electrodes to each other. By using the four electrodes 44a, 44b, 44c, and 44d and the high frequency of 90 kHz, as described above, and alternately replacing the positive electric charge of one pair of the electrodes 44a and 44d and the negative electric charge of the other pair of the electrodes negative 44b and 44c with each other with the two pairs of the electrodes being diagonally disposed respectively and with the two diagonal lines crossing with each other, the beauty instrument is capable of giving the user a feeling that the first electrode group 41 sticks to a portion of the user's skin which contacts the first electrode group owing to the stimulus imparted to the skin caused by the supply of the electric current to the skin. The beauty instrument is also capable of giving the user's skin a hyperthermic effect owing to the action of the high frequency. The beauty instrument allows the user to expect that the effect of promoting “fat combustion” and “metabolism” can be brought about owing to the hyperthermic effect.

The second electrode group 42 also brings about the same effects as the above-described effects. Therefore in a wide range of the first electrode group and the second electrode group 42, the beauty instrument is capable of giving the user the hyperthermic effect and the feeling that the second electrode group sticks to the user's skin. Although the high frequency of 90 kHz is used for the beauty instrument 10, it is possible to use different frequencies in a range of 80 kHz to 100 kHz for the beauty instrument 10 of the embodiment of the present invention.

It is possible to control the operation of the first electrode group 41 and that of the second electrode group 42 in such a way that the current supply state and the current supply suspension state are repeatingly alternated between the first electrode group 41 and the second electrode group 42 at regular time intervals. By repeating the current supply and the current supply suspension alternately, the user's skin is intermittently stimulated. Thus the user is capable of obtaining a bodily sensation that the user's skin is “tapped”.

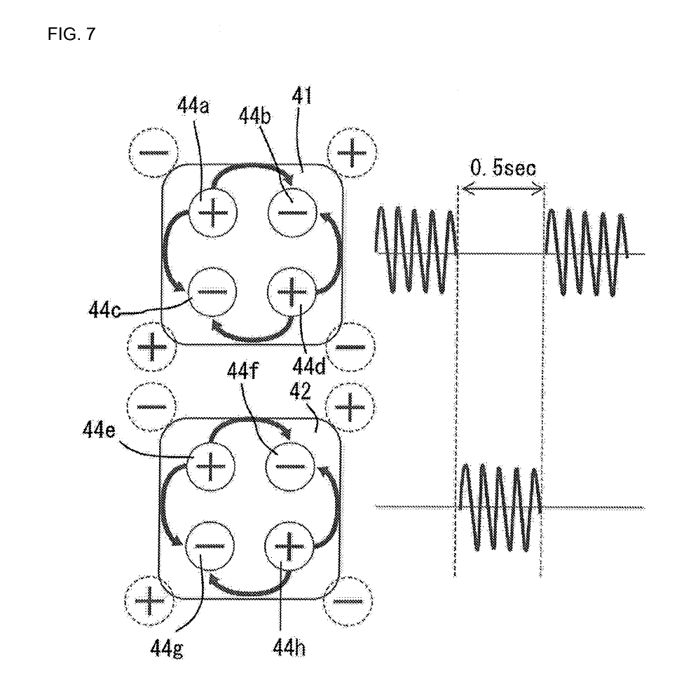

Specifically describing the above-described control method, as shown in FIG. 7, it is possible to control the operation of the first electrode group 41 and that of the second electrode group 42 in such a way that the current supply state and the current supply suspension state are alternated between the first electrode group 41 and the second electrode group 42. In the example of FIG. 7, the current supply state and the current supply suspension state are alternated between the first electrode 41 and the second electrode 42 every 0.5 seconds. By repeatingly alternating the current supply and the current supply suspension between the first electrode 41 and the second electrode 42 in the above described way, the beauty instrument is capable of bringing about the effect that the user's skin is “tapped” in a wide range. By alternating the current supply state and the current supply suspension state between the first electrode 41 and the second electrode 42 every 0.5 seconds, a portion of the user's skin to which the electric current is applied and a portion thereof in which the current supply is suspended are always alternated with each other. Thereby the beauty instrument is capable of bringing about a more rhythmical massage effect for the user's skin. It is possible to place the first electrode group 41 or the second electrode group 42 in a state in which the electric current is always supplied and the other of the first and second electrode groups in a state in which the current supply state and the current supply suspension state are alternately replaced with each other every predetermined period of time. It is possible to appropriately alter the time interval at which the current supply state and the current supply suspension state are alternately replaced with each other. It is also possible to set the time interval at which the current supply state and the current supply suspension state are alternately replaced with each other not fixedly but indefinitely.

The LEDs 50 arranged in the electrode arranging portion 16 have the following operations and effects. The LED array 51 constituting the first column consists of the blue light-emitting diode, as described above. LEDs each having a wavelength of 415 nm are used as the LEDs 50 of the LED array 51. The LED array 51 has an effect of softening the roughness of the user's skin and giving exhilaration to the skin. The LED array 52 constituting the second column consists of the red light-emitting diode, as described above. LEDs each having a wavelength of 620 nm are used as the LEDs 50 of the LED array 52. The LED array 52 is effective for users who are concerned about their fine wrinkles and users who feel fatigue on their skin and thus gives firmness to their skin and allows the skin to be shiny and elastic. The LED array 53 constituting the third column consists of the yellow light-emitting diode, as described above. LEDs each having a wavelength of 590 nm are used as the LEDs 50 of the LED array 53. The LED array 53 is effective for users who are concerned about their spots and dullness and has an effect of allowing the skin to be bright and transparent. It is possible to alter the wavelength of any of the above-described LED arrays within the range plus/minus 10 nm. In this range, the effect of the LED array is maintained.

The method of operating the beauty instrument 10 constructed as described above is as shown below. When the power button 12 disposed on the rear side of the beauty instrument 10 shown in FIG. 3 is pressed for not less than 1.5 seconds in a non-operating state, the power supply is turned on and at the same time an operation sound is emitted. Further one Lv LED 14 is turned on. One Mode LED 51 is also turned on. At this stage, a first level having a potential difference of 48 Vpp is set. At the same time, all of the LEDs of the electrode arranging portion 16 of the head part 40 are turned on. In this state, a short sound is emitted every five seconds to indicate the passage of time. When 10 minutes elapsed after the power supply is turned on, the power supply is turned off for safety. When the power button 12 is pressed in the first level state, the operation sound is emitted and the first level shifts to a second level having a potential difference of 56 Vpp. Further with one Mode LED 15 being turned on, two Lv LEDs 14 are turned on. When the power button 12 is pressed in the second level state, the operation sound is emitted, and the second level shifts to a third level having a potential difference of 64 Vpp. Further with one Mode LED 15 being turned on, three Lv LEDs 14 are turned on. When the power button 12 is pressed in the third level state, the operation sound is emitted, and the power supply is cut off. As a result, all of the LEDs are turned off.

By operating the mode button 13 with the electric current being supplied to the electrodes, the supply of the electric current from the electrodes can be controlled as described below. When the mode button 13 is briefly pressed once in the current supply state, a short sound is emitted, and the state of a mode stage 1 is set. As described above, in this mode, the current supply state and the current supply suspension state are repeatingly alternated between the first electrode group 41 and the second electrode group 42 at intervals of 0.5 seconds. Further one LV LED 14 is turned on, and one Mode LED 51 is turned on. In this case, a short sound is emitted every five seconds to indicate the passage of time. When 10 minutes pass after the power supply is turned on, the power supply is turned off for safety. When the mode button 13 is briefly pressed once in the mode stage 1, a short sound is emitted, and the mode stage 1 state shifts to a mode stage 2 state in which the current supply state and the current supply suspension state are repeatingly alternated at one-second intervals between the first electrode group 41 and the second electrode group 42. Further one Lv LED 14 is turned on, and two Mode LEDs 15 are turned on. In this case, a short sound is also emitted every five seconds to indicate the passage of time. When 10 minutes pass after the power supply is turned on, the power supply is turned off for safety. When the mode button 13 is briefly pressed once in the mode stage 2, a short sound is emitted, and the mode stage 2 shifts to a mode stage 3 state in which the current supply state and the current supply suspension state are repeatingly alternated at two-second intervals between the first electrode group 41 and the second electrode group 42. Further one Lv LED 14 is turned on, and three Mode LEDs 15 are turned on. In this case, a short sound is also emitted every five seconds to indicate the passage of time. When 10 minutes pass after the power supply is turned on, the power supply is turned off for safety.

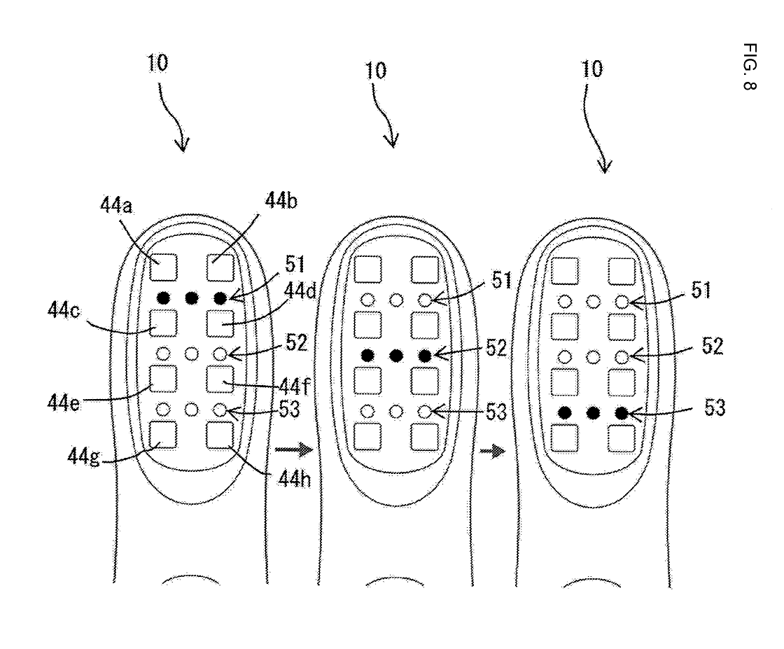

By operating the LED button 11 with the electric current being supplied to the electrodes, the emission of light of the LED 50 can be controlled as described below. When the LED button 11 is briefly pressed once in the current supply state, a short sound is emitted, and an LED stage 1 state is set. In the LED stage 1 state, all of the LEDs 50 is turned on. This state is maintained. At this time, one LV LED 14 is turned on, and one Mode LED 51 is turned on. In this case, a short sound is emitted every five seconds to indicate the passage of time. When 10 minutes pass after the power supply is turned on, the power supply is turned off for safety. When the LED button 11 is briefly pressed once in the LED stage 1, a short sound is emitted, and the LED stage 1 state shifts to an LED stage 2 state in which all of the LEDs 50 is turned on and off. This state is maintained. In this case, one Lv LED 14 is turned on, and one Mode LED 15 is turned on. In this case, a short sound is also emitted every five seconds to indicate the passage of time. When 10 minutes pass after the power supply is turned on, the power supply is turned off for safety. When the LED button 11 is briefly pressed once in the LED stage 2, a short sound is emitted, and the LED stage 2 shifts to an LED stage 3 state in which the LED arrays 51 through 53 sequentially repeat turn-on and turn-off from the upper LED array to the lower LED in order. More specifically, as shown in FIG. 8, operations of (1) only the upper LED array 51 of the head part 40 is turned on, (2) only the intermediate LED array 52 of the head part 40 is turned on, and (3) only the lower LED array 53 of the head part 40 is turned on are repeated. The LED arrays flowingly repeat turn-on and turn-off operations in a direction of an upper part of the head part 40 toward a lower part thereof. At this time, three Lv LEDs 14 are turned on, and three Mode LEDs 15 are turned on. In this case, a short sound is also emitted every five seconds to indicate the passage of time. When 10 minutes pass after the power supply is turned on, the power supply is turned off for safety.

The construction of the beauty instrument of the present invention is not limited to that of the beauty instrument 10 of the above-described embodiment. But in addition to the modifications already described, it is possible to make various modifications within the scope of the gist of the present invention. The electrodes of the present invention may have configurations other than the above-described one. For example, the electrodes of the present invention may be circular or triangular in a front view. In addition, it is possible to arrange the electrodes of the first electrode group and those of the second electrode group in configurations other than the above-described one. For example, it is possible to arrange each electrode group circularly or electrodes at the positions of the apexes of a triangle. By disposing the electrodes at the positions of the four apexes of a quadrangle as in the case of the above-described example, the electric current flows from one electrode to the adjacent two electrodes. Thereby the user can expect that the effect of the beauty instrument exerts widely on the user's skin. Particularly by disposing the electrodes of each electrode group at the positions of the apexes of a square, the user can expect that the user's skin can feel the massage effect uniformly inside the area of the square. It is possible to differentiate the arrangement configuration of the first electrode group and that of the second electrode group from each other. For example, it is possible to dispose the electrodes of the first electrode group at the positions of the apexes of the square and circularly arrange the electrodes of the second electrode group. In the above-described example, the number of the electrode groups is set to two, but not to limited to two. It is possible to set the number of the electrode groups to three or more. In the above-described example, a plurality of the electrode groups 41, 42 are arranged along the vertical direction of the beauty instrument 10. But the manner of arranging the electrode groups is not limited thereto. The electrode groups may be arranged in the lateral direction of the beauty instrument. In the case where the beauty instrument is provided with three electrode groups, the electrode groups may be arranged in a mode other than the above-described modes. For example, the electrode groups may be so arranged as to construct a triangle.

In the beauty instrument 10, it is possible to control the method of supplying the electric current to electrodes in such a way that the electric current is sequentially supplied to the electrodes 44 in a direction from the upper-end electrode to the lower-end electrode or conversely from the lower-end electrode to the upper-end electrode. It is possible to arrange a plurality of electrodes linearly or curvedly, sequentially place the arranged electrodes in the current supply state in a direction from the electrode disposed at one-end of the arranged electrodes to the electrode disposed at the other-end thereof, and thereafter sequentially place the arranged electrodes in the current supply suspension state. Thereby the user can obtain a massage effect which flows from a portion of the user's skin corresponding to the one-end side of the arranged electrodes to a portion of the user's skin corresponding to the other-end side thereof. More specifically, for example, it is possible to sequentially supply the electric current to all the electrodes 44 in the direction from the upper-end electrode to the lower-end electrode and thereafter sequentially place all the electrodes in the current supply suspension state in the direction from the upper-end electrode to the lower-end electrode. Alternatively, immediately after the electric current is supplied to the upper-end electrode 44, the upper-end electrode is placed in the current supply suspension state. Thereafter the electric current is supplied to a subsequent electrode 44 disposed immediately below the upper-end electrode. In this manner, the remaining electrodes are sequentially placed in the current supply state and the current supply suspension state.

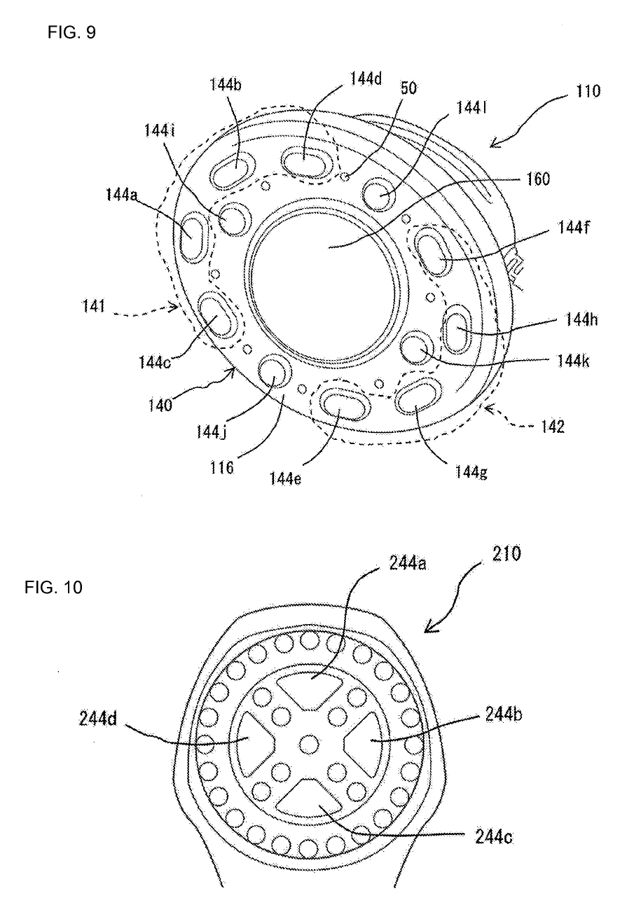

With reference to FIG. 9, a beauty instrument 110 of another embodiment of the present invention is described below. In the beauty instrument 110, a head part 140 and an electrode arranging portion 116 are elliptically formed in a front view. In accordance with the elliptical configuration thereof, electrodes 144a, 144b, 144c, 144d, 144e, 144f, 144g, and 144h are arranged elliptically along the outer edge of the electrode arranging portion 116.

In a first electrode group 141 of the beauty instrument 110, the electrodes 144a, 144b, 144c, and 144d are elliptically formed in a front view and arranged along an inner side of a circular arc part disposed near a distal end of the electrode arranging portion 116. The electrodes 144e, 144f, 144g, and 144h are also elliptically formed in a front view and arranged along an inner side of a circular arc part disposed near a proximal end of the electrode arranging portion 116. It is possible to flow the electric current of 90 kHz from these electrodes to the user's skin. As with the above-described beauty instrument 10, the operation of the four electrodes of each of the electrode groups 141 and 142 can be so controlled that a positive electric charge of one pair of the electrodes and a negative electric charge of the other pair of the electrodes (for example, the first electrode group 141 consists of a combination of the positive electrodes 144a and 144d and the negative electrodes 144b and 144c) are alternately replaced with each other 90,000 times with a diagonal line connecting the diagonally disposed two positive electrodes to each other crossing with a diagonal line connecting the diagonally disposed two negative electrodes to each other. The operation of the first electrode group 141 and that of the second electrode group 142 are controlled in such a way that the current supply state and the current supply suspension state are repeatingly alternated between the first electrode group and the second electrode group at regular time intervals. It is possible to adopt various modes regarding the time interval at which the current supply state and the current supply suspension state are alternately replaced with each other as with the above-described beauty instrument 10.

In the beauty instrument 110, at the electrode arranging portion 116, there are arranged electrodes 144i through 144l to which it is possible to supply the electric current having a frequency of about 400 kHz to about 800 kHz at which the electric current has a high deep part heating effect. At the center of the electrode arranging portion 116, there is disposed an ultrasonic generator 160 capable of generating ultrasonic waves having an output frequency of about 43 kHz to about 54 kHz and a vibration frequency of about Hz to about 90 Hz both necessary for generating cavitation. The beauty instrument 110 is capable of enhancing the beauty effect for the user's skin owing to a high deep part heating effect to be brought about by the electrodes 144i through 144l and a cavitation effect to be brought about by the ultrasonic generator 160.

A beauty instrument of still another embodiment of the present invention is described below. As shown in FIG. 10, a beauty instrument 210 is provided with four electrodes 244 disposed at the positions of the apexes of a square. In the beauty instrument 210, the operation of the four electrodes 244a through 244d can be so controlled that a positive electric charge of one pair of the electrodes and a negative electric charge of the other pair of the electrodes (for example, the combination of a pair of the positive electrodes 244a and 244d and a pair of the negative electrodes 244b and 244c) are alternately replaced with each other 90,000 times. The operation of the four electrodes 244a through 244d is so controlled that the current supply state and the current supply suspension state are repeatingly alternated at regular time intervals between a pair of the electrodes 244a and 244d and a pair of the electrodes 244b and 244c. It is possible to adopt various modes regarding the time interval at which the current supply state and the current supply suspension state are alternated as with the above-described beauty instrument 10. A similar control can be performed to allow a pair of the electrodes 244a and 244b and a pair of the electrodes 244c and 244d to have the same electric charge respectively.

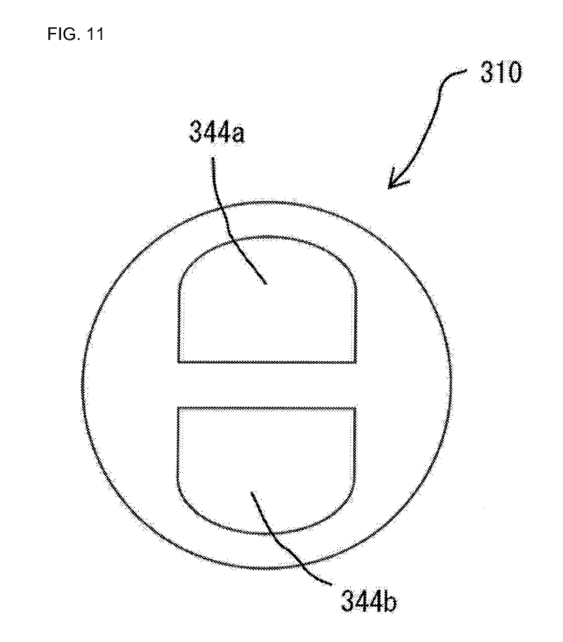

As shown in FIG. 11, it is possible to provide a beauty instrument 310 with two electrodes 344. In the beauty instrument 310, the operation of the electrodes 344a and 344b is so controlled that the current supply state and the current supply suspension state are repeatingly alternated between the electrodes 344a and 344b at regular time intervals. It is possible to adopt various modes regarding the time interval at which the current supply state and the current supply suspension state are alternately replaced with each other as with the above-described beauty instrument 10.

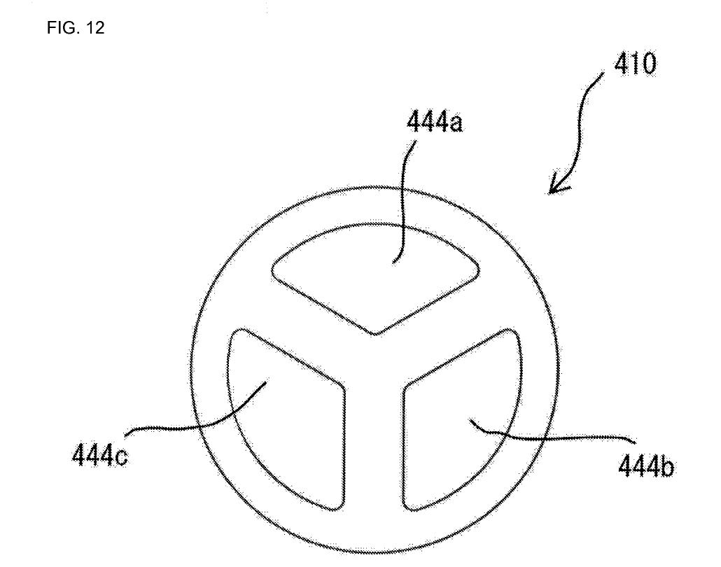

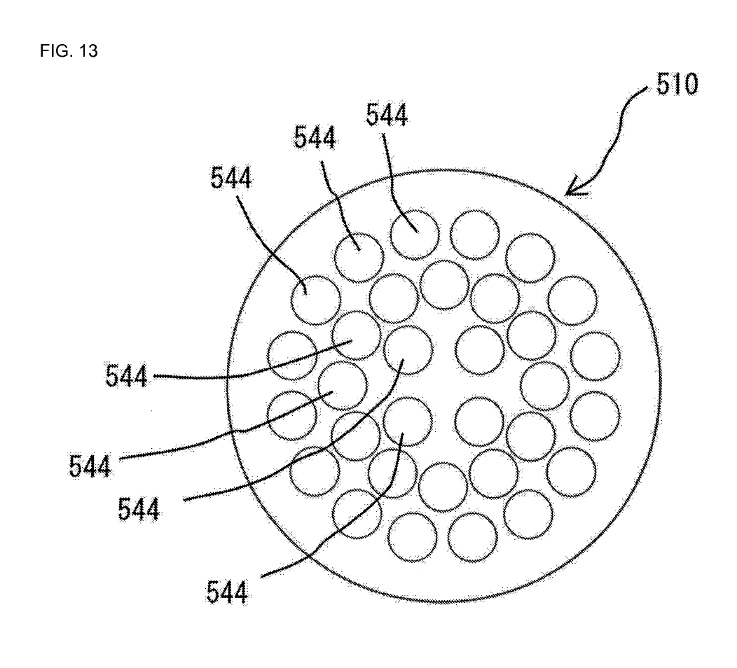

As shown in FIG. 12, it is possible to provide a beauty instrument 410 with three electrodes 444. In the beauty instrument 410, in electrodes 444a, 444b, and 444c, for example, while the electric current is supplied to the electrodes 44a and 444b for a certain period of time, the electrode 444c is placed in the current supply suspension state. Thereafter while the electric current is supplied to the electrodes 444b and 444c for a certain period of time, the electrode 444a is placed in the current supply suspension state. Thereafter while the electric current is supplied to the electrodes 444c and 444a for a certain period of time, the electrode 444b is placed in the current supply suspension state. The operation of the electrodes is so controlled that while two electrodes are conductive to each other, the remaining one electrode is placed in the current supply suspension state. The operation of the electrodes is controlled by appropriately altering the combination of the electrodes. It is possible to adopt various modes regarding the time interval at which the current supply state and the current supply suspension state are alternately replaced with each other as with the above-described beauty instrument 10.

As shown in FIG. 13, it is possible to arrange a large number of electrodes 544 at an electrode arranging portion 516 of a beauty instrument 510. In the beauty instrument 510, the electrodes 544 are roughly concentrically arranged. The operation of the electrodes 544 is so controlled that the electric current is supplied to the electrodes 544 in a direction from the electrodes disposed around the center of the electrode arranging portion 516 toward the electrodes disposed along the outer periphery thereof. More specifically, the electric current may be sequentially supplied to all the electrodes 544 in the direction from the electrodes disposed around the center of the electrode arranging portion 516 toward the electrodes disposed along the outer periphery thereof and thereafter the electrodes may be sequentially placed in the current supply suspension state in the direction from the electrodes disposed around the center of the electrode arranging portion 516 toward the electrodes disposed along the outer periphery thereof. Alternatively the electric current may be supplied to the electrodes disposed around the center of the electrode arranging portion and thereafter the electrodes may be placed in the current supply suspension state. Thereafter the electric current may be supplied to the electrodes disposed outward from and adjacent to the electrodes disposed around the center of the electrode arranging portion and thereafter the electrodes may be placed in the current supply suspension state. In this manner, the electric current may be sequentially supplied to all the electrodes, and the electrodes may be sequentially placed in the current supply suspension state. Thereby the user can obtain a massage effect which spreads from a portion of the user's skin corresponding to the center of the electrode arranging portion 516 toward a portion of the user's skin corresponding to the outer peripheral side thereof. Conversely, the operation of the electrodes 544 can be so controlled that the electric current is supplied to the electrodes in a direction from the electrodes disposed along the outer periphery of the electrode arranging portion 516 toward the electrodes disposed around the center thereof. More specifically, the electric current may be sequentially supplied to all the electrodes 544 in the direction from the electrodes disposed along the outer periphery of the electrode arranging portion 516 toward the electrodes disposed around the center thereof and thereafter the electrodes may be sequentially placed in the current supply suspension state in the direction from the electrodes disposed along the outer periphery of the electrode arranging portion 516 toward the electrodes disposed around the center thereof. Alternatively the electric current may be supplied to the electrodes disposed along the outer periphery of the electrode arranging portion and thereafter the electrodes may be placed in the current supply suspension state. Thereafter the electric current may be supplied to the electrodes disposed inward from and adjacent to the electrodes disposed along the outer periphery of the electrode arranging portion. In this manner, the electric current may be sequentially supplied to all the electrodes, and the electrodes may be sequentially placed in the current supply suspension state.

In addition to the above-described examples of the electrodes, in a beauty instrument having a massage roller and a beauty instrument having a ball, the roller and the ball may be provided with electrodes. The operation of the electrodes is controlled in such a way that a current supply state and a current supply suspension state are alternated among the electrodes in a manner similar to the above-described manner.

### INDUSTRIAL APPLICABILITY

The beauty instrument of the present invention can be widely utilized for the beauty of user's hands, arms, legs, face, skin, and the like.

### EXPLANATION OF REFERENCE SYMBOLS AND NUMERALS

10: beauty instrument, 11: LED button, 12: power button, 13: mode button, 14: LVLED, 15: Mode LED, 16: electrode arranging portion, 20: gripping part, 30: intermediate part, 31: power supply substrate, 32: converter, 34: rechargeable battery, 35: microcomputer, 36: LED driver, 37: power booster, 38: RF power controller, 40: head part, 41: first electrode group, 42: second electrode group, 44a, 44b, 44c, 44d, 44e, 44f, 44g, and 44h: electrode, 50: LED, 51, 52, and 53: LED array, 110: beauty instrument, 116: electrode arranging portion, 140: head part, 141: first electrode group, 142: second electrode group, 144a, 144b, 144c, 144d, 144e, 144f, 144g, 144h, 144i, 144j, 144k, and 144l: electrode, 160: ultrasonic generator, 210: beauty instrument, 244a, 244b, 244c, and 244d: electrode, 310: beauty instrument, 344a, 344b: electrode, 410: beauty instrument, 444a, 444b, and 444c: electrode, 510: beauty instrument, 516: electrode arranging portion, and 544: electrode

1. A beauty instrument comprising a plurality of electrodes and being capable of applying high-frequency waves to user's skin from said electrodes thereof, wherein a plurality of said electrodes is continuously arranged, and an operation of a plurality of said electrodes can be so controlled that a current supply state and a current supply suspension state are alternated among a plurality of said electrodes every predetermined period of time.

2. A beauty instrument comprising a plurality of electrodes capable of applying high-frequency waves to user's skin from said electrodes thereof, wherein a plurality of said electrodes is arranged by separating said electrodes into a plurality of electrode groups with said electrodes being continuous with each other, and an operation of said electrode groups can be so controlled that a current supply state and a current supply suspension state are alternated among a plurality of said electrode groups every predetermined period of time.

3. The beauty instrument according to claim 1, wherein a number of said electrodes is 4n (n=1 or more), and four electrodes of each electrode group are disposed at positions of apexes of a quadrangle, and an operation of said four electrodes disposed at said positions of said apexes of said quadrangle can be so controlled that a positive electric charge of one pair of said electrodes and a negative electric charge of said other pair of said electrodes are alternately replaced with each other with a diagonal line connecting said positive diagonally disposed two electrodes to each other crossing with a diagonal line connecting said negative diagonally disposed two electrodes to each other.

4. The beauty instrument according to claim 2, wherein a number of a plurality of electrodes of each of said electrode groups is set to four, and said four electrodes are disposed at positions of apexes of a quadrangle, and an operation of said four electrodes disposed at said positions of said apexes of said quadrangle can be so controlled that a positive electric charge of one pair of said electrodes and a negative electric charge of said other pair of said electrodes are alternately replaced with each other with a diagonal line connecting said diagonally disposed two positive electrodes to each other crossing with a diagonal line connecting said diagonally disposed two negative electrodes to each other.

5. The beauty instrument according to claim 2 formed substantially cylindrically, and at least two of said electrode groups are adjacently and continuously arranged along a longitudinal direction of said beauty instrument.

6. The beauty instrument according to claim 1, wherein a plurality of said electrodes is linearly arranged, and an operation of said linearly arranged electrodes can be so controlled that said electrodes are sequentially placed in a current supply state in a direction from said electrode disposed at one-end side of said linearly arranged electrodes toward said electrode disposed at said other-end side thereof and thereafter sequentially placed in a current supply suspension state.

7. The beauty instrument according to claim 1, wherein a plurality of said electrodes is arranged in a direction from a center of an electrode arranging portion toward an outer periphery thereof, and an operation of a plurality of said arranged electrodes can be so controlled that said arranged electrodes are sequentially placed in a current supply state in a direction from said electrodes disposed at a center of said arranged electrodes toward said electrodes disposed along an outer periphery thereof and thereafter sequentially placed in a current supply suspension state.

8. The beauty instrument according to claim 1, wherein a plurality of said electrodes is arranged in a direction from an outer periphery of an electrode arranging portion toward a center thereof, and an operation of a plurality of said arranged electrodes can be so controlled that said arranged electrodes are sequentially placed in a current supply state in a direction from said electrodes disposed along an outer periphery of said arranged electrodes toward said electrodes disposed at a center thereof and thereafter sequentially placed in a current supply suspension state.

9. The beauty instrument according to claim 1, wherein an operation of said electrodes can be so controlled that a current supply state and a current supply suspension state are alternated among said electrodes at time intervals of 0.20 seconds to 3.00 seconds.

10. The beauty instrument according to claim 1, wherein an operation of said electrodes can be so controlled that a current supply state and a current supply suspension state are alternated among said electrodes at time intervals of 0.25 seconds to 1.00 second.

11. The beauty instrument according to claim 1 comprising an LED configured to irradiate a user's skin with light.

12. The beauty instrument according to claim 11, wherein said LED can be so controlled that simultaneously with a supply of an electric current from said electrodes to said LED, said LED is capable of irradiating user's skin with light.

13. The beauty instrument according to claim 11, wherein said LED consists of a blue light-emitting diode, a red light-emitting diode, and a yellow light-emitting diode.

14. The beauty instrument according to claim 1 further comprising an electrode to which an electric current having a frequency of about 400 kHz to about 800 kHz at which a high deep part heating effect is obtained.

15. The beauty instrument according to claim 1 further comprising an ultrasonic generator configured to generate ultrasonic waves having an output frequency of about 43 kHz to about 54 kHz and a vibration frequency of about 70 Hz to about 90 Hz for generating cavitation.
